(19) United States
(12) Patent Application Publication
(10) Pub. No.: US 2013/0345470 A1
(43) Pub. Date: Dec. 26, 2013

(54) Purification of 3-Hydroxypropionic Acid from Crude Cell Broth and Production of Acrylamide

(71) Applicants: Robert Tengler, Longmont, CO (US); David DeCoster, Lyons, CO (US)

(72) Inventors: Robert Tengler, Longmont, CO (US); David DeCoster, Lyons, CO (US)

(73) Assignee: OPX Biotechnologies, Inc., Boulder, CO (US)

(21) Appl. No.: 13/527,799

(22) Filed: Jun. 20, 2012

(51) Int. Cl. C07C 51/42; C07C 67/08; C07C 231/02; C12M 1/00; C07C 69/675; C07C 235/04; C07C 233/09; C07C 59/01; C07C 231/12

(52) U.S. Cl. 560/179; 562/513; 562/579; 564/134; 564/205; 564/201; 564/204; 435/294.1

(57) ABSTRACT

A process for producing high purity 3-hydroxypropionic acid from a fermentation cell broth is described. The 3-hydroxypropionic acid can be converted to a variety of products, such as acrylamide, 3-hydroxypropionic esters, acrylic esters, and 3-HP amide. This process features a high degree of product flexibility, limited or no solvent recycle, discrete waste streams, an efficient water removal process, and efficient recovery of products and solvents with proven and scalable equipment.

### BACKGROUND

Many basic chemical building blocks, such as alcohols, carboxylic acids, and olefins, are derived from petroleum. With increasing acceptance that petroleum hydrocarbon supplies are decreasing and their costs are increasing, there is a growing trend of developing and improving industrial microbial systems for production of chemicals and fuels. Such industrial microbial systems could at least partially replace the use of petroleum hydrocarbons for production of certain chemicals.

One candidate chemical for biosynthesis in industrial microbial systems is 3-hydroxypropionic acid (“3-HP”, CAS No. 503-66-2). 3-HP is a highly valuable building block used in the production of a number of chemicals, such as acrylic acid, acrylates, and acrylamide, which can be further converted to a wide range of industrial and consumer products.

Currently there is an interest in microbial production of 3-HP and further use thereof. However, microbial production of 3-HP generates aqueous product streams that are dilute and contain a variety of impurities and byproducts. There remains a need for improved methods of purifying and converting 3-HP to other chemical and consumer products.

### SUMMARY OF THE INVENTION

In accordance with the present invention, there is provided a process to produce high purity 3-hydroxypropionic acid (3-HP) from a fermentation broth, comprising (a) removing a substantial amount of water from the fermentation broth to give a concentrated fermentation broth; and (b) extracting the 3-HP from the concentrated fermentation broth with an organic solvent, wherein the organic solvent has a boiling point of less than 170° C. The process may additionally comprise a distillation step. The 3-HP thus obtained may have purity higher than 90% or even higher than 95%.

The fermentation broth may contain a substantial amount of whole cells. On the other hand, the fermentation broth may be substantially whole-cell free. If desired, a substantial amount of whole cells are removed with a centrifuge.

Techniques for removing water are well known in the art. For example, water can be removed by evaporation. Evaporation can be conducted in a variety of ways, such as heating, and/or under reduced pressure. In one aspect, the evaporation is performed under reduced pressure. In a further aspect, the pressure is a range of 20-60 mbar. In another aspect, the evaporation is performed at a temperature higher than 30° C. In a further aspect, the temperature is a range of 60-150° C. Evaporation can also be conducted with a variety of evaporators, for example, evaporators using mechanical recompression methods and thin film evaporators. Prior to or during the water removing process, solids may precipitate out of the broth. The precipitated solids can be separated from the rest of the material. The separation can be conducted by, for example, filtration. If desired, the solid may be washed with an organic solvent to improve the recovery of 3-HP. In addition, water can be removed by distilling off a water-containing distillate. For example, an azeotropic distillation with an organic solvent such as toluene can be performed. Alternatively, an alcohol solvent may be used in the distillation step.

In accordance with the present invention, the process may further comprise adjusting pH of the concentrated fermentation broth to give an acidic concentrated fermentation broth. In some embodiments, the pH is adjusted with an inorganic acid. In a further embodiment, the inorganic acid is sulfuric acid or phosphoric acid. During the pH adjusting process, solids may precipitate out of the concentrated acidic broth. The solids can be separated from the rest of the material, for example, by filtration. If desired, the solids may be washed with an organic solvent. The organic solvent may be the same solvent as used in the step b.

The organic solvent used in the extraction and washing steps may be alcohols, aldehydes, ketones and ethers. Non-limiting examples of alcohols are C₁-C₁₂ alcohols, for example, methanol, ethanol, n-propanol, iso-propanol, n-butanol, iso-butanol, tert-butanol, 2-hexanol, n-octanol and their isomers. Non-limiting examples of aldehydes are methyl aldehyde, ethyl aldehyde, propyl aldehyde, butyl aldehyde and their isomers. Non-limiting examples of ketones are acetone and methyl ethyl ketone. Non-limiting examples of ethers are tetrahydrofuran, 2-methyltetrahydrofuran and 1,2-dimethoxyethane.

The choice and amount of the solvent may affect the extraction efficiency of step b. If properly chosen, about 85%, 86%, 87%, 88%, 89%, 90%, 91%, 92%, 93%, 94%, 95%, 96%, 97%, 98%, 99% or more of total 3-HP can be recovered from the fermentation broth. In addition, the choice of the solvent may depend upon the final product of the process. For example, if a 3-HP ester or acrylic ester is desired, an alcohol solvent is preferred. The 3-HP alcohol extract obtained from step b may be heated to generate a 3-HP ester. If desired, a catalytic amount of sulfuric acid may be added to speed up the esterification reaction. The 3-HP ester can be purified, for example, by distillation. The process may further comprise a dehydration step to produce an acrylic ester. When an alcohol solvent is used to produce a 3-HP ester, the alcohol solvent serves as an extracting solvent and as a reactant for the esterification reaction. This could lead to a process with minimal or no solvent removal. In some embodiments, the amount of alcohol used may be less than two equivalents of the total amount of the 3-HP present in the fermentation broth. In other embodiments, the amount of alcohol used may be about one equivalent of the total amount of the 3-HP present in the fermentation broth.

In accordance with the present invention, there is provided a process of producing 3-HP amide, comprising (a) removing a substantial amount of water from a fermentation broth to give a concentrated fermentation broth; (b) extracting the 3-HP from the concentrated fermentation broth with an organic solvent; and (c) converting the 3-HP to 3-HP amide. Step c can be carried out in a variety of ways. For example, esterification of 3-HP gives a 3-HP ester; amidation of the 3-HP ester generates 3-HP amide. The amidation reaction may be performed with ammonia gas or an ammonium ion.

The present disclosure provides a method of producing acrylamide, comprising: (a) providing a fermentation broth comprising 3-HP, or salt thereof; (b) removing a substantial amount of water from said fermentation broth to give a concentrated fermentation broth; (c) extracting said 3-HP from said concentrated fermentation broth with an organic solvent; and (d) converting said 3-HP to acrylamide.

3-HP, 3-HP amide, acrylamide, 3-HP ester, acrylic ester and other downstream products are useful intermediates for a variety of chemicals and consumer products, for example, acrylic acid and acrylic ester-based polymers and copolymers, diaper, feminine hygiene product, adult incontinence product, paint, coating, ink and thickening agent. Thus, the present disclosure provides a method of producing a 3-HP-based product, comprising: (a) producing 3-HP according to the method described herein; and (b) converting the 3-HP into a 3-HP-based product.

Additionally, the present disclosure provides a system, comprising: (a) a fermenter; (b) an evaporator; (c) a centrifuge; (d) an extractor; (e) a reactive distillation apparatus; (f) an esterification tank; and (g) an amidation reactor. The fermenter may comprise a fermentation broth having a 3-HP concentration of at least 2, 3, 4, 5, 6, 7, 8, 9, 10, 11, 12, 13, 14, 15, 20, or 30 g/L. The 3-HP concentration may be in a range of 2-10, 3-12, 4-15, 5-20, 6-25 or 7-30 g/L. The centrifuge may comprise a liquid phase having a 3-HP concentration of at least 2, 3, 4, 5, 6, 7, 8, 9, 10, 11, 12, 13, 14, 15, 20, or 30 g/L. The 3-HP concentration in the centrifuge may be in a range of 2-10, 3-12, 4-15, 5-20, 6-25 or 7-30 g/L. The system may be configured in such a way that the evaporator is upstream of the extractor. The system may further comprise a dehydration reactor. The system may be applied to, for example, production of acrylamide from a fermentation broth containing 3-hydroxypropionic acid. The system may process at least 100, 200, 300, 400, 500, 600, 700, 800, 900, 1,000, 2,000, 3,000, 4,000, 5,000, 6,000, 7,000, 8,000, 9,000, or 10,000 liters of fermentation broth per day. The system may process 100-200,000 liters of fermentation broth per day.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The novel features of the invention are set forth with particularity in the claims. A better understanding of the features and advantages of the present invention will be obtained by reference to the following detailed description that sets forth illustrative embodiments, in which the principles of the invention are utilized, and the accompanying drawings of which:

FIG. 1 illustrates a process of obtaining high purity 3-HP and production of 3-HP ester, 3-HP amide and acrylamide embodying principles of the present invention.

FIG. 2 illustrates a system for producing acrylamide embodying principles of the present invention.

### DETAILED DESCRIPTION

The present disclosure provides processes for the purification of 3-HP from a crude fermentation broth and downstream processes to produce other chemical products, for example, acrylamide, 1,3-propanediol, acrylic acid, 3-HP esters, 3-HP amide, and acrylic esters. A variety of industrial and consumer products can be further derived from chemical products produced from 3-HP.

Various teachings and examples herein describe an efficient process for purifying 3-HP from a crude fermentation broth and the downstream processes for producing acrylamide and other products. Features of various approaches described herein include: (1) product flexibility from a single feedstock; (2) limited or no solvent recycle; (3) discrete waste streams; (4) an efficient water removal process; and (5) efficient recovery of products and solvents with proven and scalable equipment.

The production of 3-HP by microbial systems has been described in, for example, WO 2010/011874, US application 2010/0021978, U.S. Pat. No. 6,852,517, US application 2009/0325248 and US application 2011/0244575, and are herein incorporated by reference. Common to microbial systems described, water is a part of the production system. Typically, a fermentation broth containing 3-HP, water, whole cells and other impurities is produced after fermentation. Separation of 3-HP from these substances may be required for downstream uses of 3-HP to produce other chemical and consumer products.

However, extraction of 3-HP from an aqueous medium represents a challenge for this process. With only 3 carbons, a carboxyl group and a hydroxyl group, 3-HP is highly hydrophilic. Therefore, the extraction efficiency of 3-HP from water with an organic solvent is usually low. Traditional organic acid isolation approaches often rely on a biphasic liquid-liquid extraction process to isolate the acid. For example, U.S. Pat. No. 7,279,598 describes a process for separating and recovering 3-HP using counter current extraction of the aqueous solution with ethyl acetate and is herein incorporated by reference for these teachings.

In accordance with the present invention, there is provided a process to produce high purity 3-hydroxypropionic acid (3-HP) from a fermentation broth, comprising (a) removing a substantial amount of water from the fermentation broth to give a concentrated fermentation broth; and (b) extracting the concentrated fermentation broth with an organic solvent.

The process of the present disclosure is useful for recovery of 3-HP from a microbial fermentation broth. The process of the present disclosure is particularly useful for recovery of 3-HP produced via a microbial fermentation process in which 3-HP needs to be recovered or purified at some point in the fermentation or manufacturing process. Fermentation broth used in the present disclosure is not limited to any particular organism, pathways, carbon source, composition, nature of impurities, the amount of whole cells and initial 3-HP concentration after fermentation. Fermentation broth used in the present disclosure may contain a substantial amount of whole cells or may be substantially whole-cell free. If desired, a substantial amount of whole cells can be removed with a centrifuge.

Methods of removing water are well-known and include, for example, distillation and evaporation. A variety of equipment for the above mentioned methods is commercially available. Distillation can be conducted at certain temperature and/or pressure. The choice of a particular temperature and/or pressure may depend on factors, such as, the size of distillation equipment, the volume of fermentation broth and the initial concentration of 3-HP. In some embodiments, the temperature may be higher than 30° C. In a further embodiment, the temperature is in a range of 60-150° C. In some embodiments, the evaporation is performed at atmospheric or under reduced pressure. In a further embodiment, the pressure is a range of 20-60 mbar.

Water can also be removed by using azeotropic distillation. Non-limiting examples of solvent for azeotropic distillation include benzene, toluene, pentane, cyclohexane, hexane, heptane, isooctane, acetone, alcohols, and diethyl ether. Typically in azeotropic distillation, the distillation of a water-containing distillate removes water.

Upon removing a substantial amount of water from the fermentation broth, a concentrated fermentation broth is obtained. The concentrated fermentation broth may still contain water, which can be used directly in the extraction step (step b). Structurally, 3-HP contains a carboxylic acid group. Carboxylic acid usually exists in two forms in aqueous media, acid form (COOH) and ionized form (COO⁻). The equilibrium between the two forms is usually dictated by pH of the aqueous media. The acid form usually has higher solubility in an organic solvent than its ionized form. Thus, acidification of the concentrated fermentation broth may be needed to enhance the extraction efficiency in step b. The acid used for adjusting pH may be a mineral acid. Non-limiting examples of mineral acid include sulfuric acid, hydrochloric acid, polyphosphoric acid, phosphoric acid and hydrobromic acid.

A variety of suitable organic solvent can be used in the present invention. Without being limiting, suitable organic solvents include, for example, alcohols, ketones, aldehydes and ethers. Non-limiting examples of alcohols are C₁-C₁₂ alcohols, for example, methanol, ethanol, n-propanol, isopropanol, n-butanol, iso-butanol, tert-butanol, 2-hexanol, n-octanol and their isomers. Non-limiting examples of aldehydes are methyl aldehyde, ethyl aldehyde, propyl aldehyde, butyl aldehyde and their isomers. Non-limiting examples of ketones are acetone and methyl ethyl ketone. Non-limiting examples of ethers are tetrahydrofuran, 1,2-dimethoxyethane and 2-methyltetrahydrofuran. In the present invention, water-soluble alcohols, such as, methanol and ethanol, are suitable solvents for extracting 3-HP. However, in the traditional biphasic liquid-liquid extraction methods, the use of methanol and ethanol as solvent is limited by their miscible nature with water. Even in cases phase separation does occur, the extraction efficiency is usually low because the aqueous layer contains a substantial amount of methanol or ethanol.

In some embodiments, the organic solvent used in the present invention has a boiling point lower than that of 3-HP or 3-HP salt in the fermentation broth. In some further embodiments, the organic solvent used in the present invention has a boiling point of less than 170° C. at 1 atmosphere pressure. In some further embodiments, the organic solvent has a boiling point equal to or lower than that of hexanol. In various embodiments, during the distillation process to purify 3-HP or an ester or amide thereof, the organic solvent is distilled and collected prior to the collection of 3-HP, or its ester or amide thereof.

The present invention also provides a process to produce 3-HP ester from a fermentation broth, comprising (a) removing a substantial amount of water from the fermentation broth to give a concentrated fermentation broth; (b) extracting the 3-HP from the concentrated fermentation broth with an alcohol solvent; and (c) refluxing or distilling the 3-HP containing extract to generate the 3-HP ester.

The process of producing the 3-HP ester may require acid in step c. The acid may come from different steps of the process. For example, the concentrated fermentation broth may be acidified prior to extraction of step b to give a concentrated fermentation broth which is acidic. Depending upon the choice of extracting solvent and property of the acidic concentrated fermentation broth, the 3-HP extract from step b may contain enough acid for the esterification reaction in step c. In cases extra acid is needed, acid can be added prior or during step c. The acid used for acidification of the fermentation broth and the acidification in step c may be the same or different. The acid is selected from the group consisting of a nitrogen, halogen, sulfur and phosphorous acid. Non-limiting examples of acid include sulfuric acid, hydrochloric acid, phosphoric acid, polyphosphoric acid, hydrobromic acid and mixture thereof.

The present invention also provides a process of producing 3-HP amide from a fermentation broth, comprising (a) removing a substantial amount of water from the fermentation broth to give a concentrated fermentation broth; (b) extracting the 3-HP from the concentrated fermentation broth with an alcohol solvent; and (c) converting the 3-HP to 3-HP amide. The conversion of 3-HP to 3-HP amide generally includes the steps of ester formation and amidation. For example, synthesis of 3-HP amide may be accomplished by refluxing the 3-HP containing extract to generate a 3-HP ester; and carrying out an amidation reaction with, for example, ammonia gas or an ammonium ion, to generate the 3-HP amide.

The present invention also provides a process of producing acrylamide from a fermentation broth, comprising (a) removing a substantial amount of water from the fermentation broth to give a concentrated fermentation broth; (b) extracting the 3-HP from the concentrated fermentation broth with an alcohol solvent; and (c) converting the 3-HP to acrylamide. The conversion of 3-HP to acrylamide generally includes steps of amide formation and dehydration. The amide formation step may require activation of the carboxylic acid group and amidation. These steps may be carried out in any order. For example, synthesis of acrylamide may be accomplished by refluxing the 3-HP containing extract to generate a 3-HP ester; carrying out an amidation reaction to generate the 3-HP amide; and dehydration to give acrylamide.

Dehydration converts a carbon-carbon single bond to a carbon-carbon double bond and produces a water molecule. The dehydration may take place in the presence of a suitable homogeneous or heterogeneous catalyst. Suitable dehydration catalysts include acids, bases and oxides. Non-limiting examples of acids are H₂SO₄, HCl, titanic acids, metal oxide hydrates, metal sulfates (MSO₄, where M=Zn, Sn, Ca, Ba, Ni, Co, or other transition metals), metal oxide sulfates, metal phosphates (e.g., M₃(PO₄)₂, where M=Ca, Ba), metal phosphates, metal oxide phosphates, carbon (e.g., transition metals on a carbon support), mineral acids, carboxylic acids, salts thereof, acidic resins, acidic zeolites, clays, SiO₂/H₃PO₄, fluorinated Al₂O₃, phosphotungstic acids, phosphomolybdic acids, silicomolybdic acids, silicotungstic acids and carbon dioxide. Non-limiting examples of bases are NaOH, ammonia, polyvinylpyridine, metal hydroxides, Zr(OH)₄, and substituted amines. Non-limiting examples of oxides are TiO₂, ZrO₂, Al₂O₃, SiO₂, ZnO₂, SnO₂, WO₃, MnO₂, Fe₂O₃, V₂O₅.

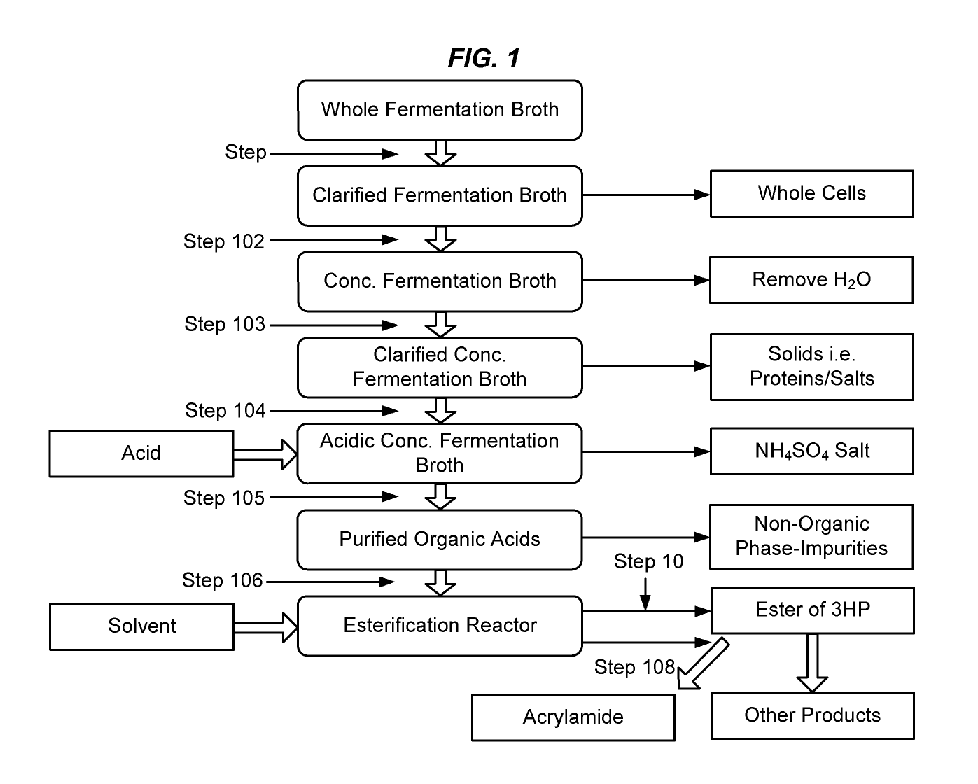

FIG. 1 depicts one scheme illustrating the isolation of high purity 3-HP from a fermentation broth and the downstream production of 3-HP ester and acrylamide embodying the principles of the present invention. It is understood that the order of steps in FIG. 1 is illustrative and may be altered based on a variety of factors, for example, the scale of the purification, the impurity in the fermentation broth, the solvent, and the equipment used for fermentation and purification. In addition, any of the steps may be repeated or some of the steps may be eliminated. The process described in FIG. 1 should not limit the scope of the present invention.

Turning to FIG. 1, a whole fermentation broth from fermentation may be transferred from a fermenter to separation equipment and subjected to a separation step 101. The concentration of 3-HP in the fermentation broth may be at least 2, 3, 4, 5, 6, 7, 8, 9, 10, 11, 12, 13, 14, 15, 20, or 30 g/L. Step 101 may allow the separation of a substantial amount of whole cells from the whole fermentation broth to give a clarified fermentation broth. The amount of whole cell removed from the whole fermentation broth may be at least 90%, at least 91%, at least 92%, at least 93%, at least 94%, at least 95%, at least 96%, at least 97%, at least 98%, or at least 99% of the initial amount of whole cells. A variety of techniques may be employed for the separation. For example, without being limiting, techniques utilizing gravitation force or centrifugal force may be employed. Further, the whole fermentation broth may be left undisturbed for a selected period of time to achieve the desired separation. The selected period of time may be about 2 h, about 4 h, about 6 h, about 8 h, about 12 h, about 18 h, about 24 h, about 36 h, about 2 days, about 3 days, about 4 days, about 5 days, about 6 days, or about 1 week. In various embodiments, the whole fermentation broth is subjected to centrifugal force to remove whole cells. The speed and the length of the centrifugation can be controlled. The centrifugation may be run at a speed of at least 250 G, at least 500 G, at least 1,000 G, at least 1,500 G, at least 2,000 G, at least 2,500 G, at least 3,000 G, at least 3,500 G, at least 4,000 G, at least 4,500 G, at least 5,000 G, at least 5,500 G, at least 6,000 G, at least 6,500 G, at least 7,000 G, at least 8,000 G, at least 9,000 G, or at least 10,000 G. The centrifuge may be run at a speed of 250-7000 G. In some embodiments, the centrifugation may last at least 5 minutes, at least 10 minutes, at least 15 minutes, at least 20 minutes, at least 25 minutes, at least 30 minutes, at least 35 minutes, at least 40 minutes, at least 50 minutes, at least 60 minutes, at least 80 minutes, at least 100 minutes, at least 120 minutes, at least 140 minutes, at least 160 minutes, at least 180 minutes, at least 200 minutes, at least 250 minutes, at least 300 minutes, at least minutes 400 minutes, or at least 500 minutes. In some cases, the centrifugation may last 5-500 minutes.

In some embodiments, the centrifugation step may keep cells intact, which may make the addition of flocculating agents to achieve clarity unnecessary. In certain other embodiments, flocculating agents may be added. Flocculating agents reduce zeta potential of charged particles and thus facilitate the aggregation (floc formation) of the particles. Non-limiting examples of flocculating agents may include, but are not limited to, neutral electrolytes such as KCl, NaCl, calcium salts, alum, sulfate, citrates, and phosphates salts. The amount of flocculating agent added to the whole broth 101 may be about 0.0001 g/L, about 0.001 g/L, about 0.01 g/L, about 0.05 g/L, about 0.1 g/L, about 0.2 g/L, about 0.3 g/L, about 0.4 g/L, about 0.5 g/L, about 0.6 g/L, about 0.7 g/L, about 0.8 g/L, about 0.9 g/L, about 1 g/L, about 2 g/L, about 3 g/L, about 4 g/L, about 5 g/L, about 6 g/L, about 7 g/L, or about 10 g/L. The amount of flocculating agent added to the whole broth 101 may be 0.01-10 g/L. The amount of flocculating agent may depend on the types of the agent used.

After reducing the amount of whole cells in the whole fermentation broth, a clarified fermentation broth is generated. To facilitate the separation of 3-HP, a substantial amount of water in the clarified fermentation broth is removed in step 102. A variety of techniques well known in the art may be used to remove water, for example, evaporation, boiling and distillation. The amount of water being removed may be at least at least 50%, at least 60%, at least 70%, at least 80%, at least 90%, at least 91%, at least 92%, at least 93%, at least 94%, at least 95%, at least 96%, at least 97%, at least 98%, or at least 99% of the initial amount. Some water maybe chemically bound and may not be easily removed under evaporative conditions. In some embodiments, the water is removed by evaporation. It is well-known in the art that evaporation of water can be conducted in a variety of ways, for example, by reducing the pressure and/or heating the solution. In some embodiments, the pressure is in a range of an ambient atmospheric pressure and about 0.1 mbar. In some embodiments, the pressure is in a range of 20-80 mbar. In some other embodiments, the pressure is an ambient atmospheric pressure. The temperature for water removal may be in a range of 25° C. and 150° C., depending upon the pressure, the scale of the reaction and the equipment used for the distillation. In certain other embodiments, the evaporation of water may be achieved by mechanical recompression methods. Mechanical recompression evaporators have been described in the art. In this context, U.S. Pat. Nos. 4,303,468 and 4,581,829 are herein incorporated by reference for their teaching. In these systems, vapor generated from an evaporator is compressed to a higher pressure so that it can be condensed with an evaporator heat exchanger. Since the temperature of the compressed vapor is higher than the boiling point of the solution, heat flows from the vapor to the solution and more vapors are generated, thus improving energy efficiency and eliminating the need of cooling water. Mechanical recompression evaporator is commercially available, for example, from Swenson Technology, Inc. The mechanical recompression methods may be more economical to remove water than evaporation.

In order to achieve high extraction efficiency, a high concentration of 3-HP would be desirable. Thus, the concentration fold would depend on the titer of the fermentation broth. For example, in order to achieve a concentration of 750 g/L 3-HP, broth of 50 g/L 3-HP needs to be concentrated about 15 fold. On the other hand, a fermentation broth of 100 g/L 3-HP would only need to be concentrated 7.5 fold. The distilled water can be re-used in the fermentation process.

Other dewatering methods may be employed solely or in any combination, including evaporation, drying and azeotropic distillation.

After removing water from the clarified fermentation broth, a concentrated fermentation broth is obtained. The concentration of 3-HP in the concentrated fermentation broth may be at least 50 g/L, or at least 60, 70, 80, 90, 100, or 110 g/L. In some cases, the concentration of 3-HP in the concentrated fermentation broth may be in a range of 50-200 g/L. In the concentrated fermentation broth, 3-HP may remain a liquid in the mixture, and impurities, such as salts and proteins, may become insoluble. The impurities may be separated in step 103 by taking advantage of their immiscible nature with 3-HP. Step 103 may be carried out during and/or after removing water. In some embodiments, the impurities are separated during concentration using a scraped drum evaporator. In some other embodiments, the impurities are separated after the concentration using centrifugation or filtration. If desired, the solids can be washed with an organic solvent to improve recovery of 3-HP.

After the separation, a clarified concentrated fermentation broth is obtained. There may be some residual impurities in the clarified concentrated fermentation broth. The amount of impurities may be at least at least 1.0%, at least 1.5%, at least 2.0%, at least 2.5%, at least 3.0%, at least 3.5%, at least 4.0%, at least 4.5%, at least 5.0%, at least 5.5%, at least 6.0%, at least 6.5%, at least 7.0%, at least 7.5%, at least 8.0%, at least 8.5%, at least 9.0%, at least 9.5%, at least 10.0%, at least 11.0%, at least 12.0%, at least 13.0%, at least 14.0%, or at least 15.0% of the initial amount of impurities.

The clarified concentrated fermentation broth may be neutral or acidic. The pH may be in a range of 3.5-4.0, 4.0-4.5, 4.5-5.0, 5.0-5.5, 5.5-6.0, 6.0-6.5, 6.5-7.0. Since 3-HP is a carboxylic acid, an acidification step 104 may be needed. Acidification of the clarified concentrated fermentation broth will shift 3-HP to the acid form, increasing solubility of 3-HP in an extraction solvent. A variety of external acids may be used to adjust the pH. The acid may be an organic or an inorganic acid. Inorganic acid may include, for example, hydrochloric acid, hydrobromic acid, sulfuric acid, nitric acid, phosphoric acid, polyphosphoric acid, and mixture thereof. In some embodiments, sulfuric acid is used. In some other embodiments, phosphoric acid is used. The choice of acid may dictate the resulting salts which then fall out of solution. These salts may be significant in that an equal molar ratio of salt to 3-HP and other organic acids is expected.

Upon acidification, an acidic concentrated fermentation broth is obtained. The pH of the broth may be in a range of below 0, 0-1, 1.0-1.5, 1.5-2.0, 2.0-2.5, 2.5-3.0, 3.0-3.5, 3.5-4.0, 4.0-4.5, 4.5-5.0, 5.0 to 5.5. Upon acidification, a salt may be precipitated out of the broth. In some embodiments, the salt is an ammonium salt of the chosen inorganic acid. The salt and the liquid may be separated in step 105 by methods, for example, filtration.

Salts and other solids may be formed upon acidification. The resulting solids can then be removed during an optional filtration step. A wash step of the solids may be needed due to the amount of the 3-HP remaining in the interstitial space of the solid cake. The amount of residual 3-HP in the solid cake may be at least 5%, at least 10%, at least 15%, at least 20%, at least 25%, at least 30%, at least 35%, at least 40%, at least 45%, or at least 50% of total 3-HP. The wash may be carried out with the organic solvent used in the extraction step, such as an alcohol. For example, an alcohol may be selected from C₁-C₁₂ aliphatic alcohols, for example but not limited to, methanol, ethanol, 1-propanol, 1-butanol, isopropyl alcohol, isobutyl alcohol, t-butanol, 1-octanol, 1-hexanol, 2-hexanol, and their isomers. A selected alcohol may be an alcohol which is miscible or immiscible with water. This is in contrast to traditional biphasic extraction techniques in which water-immiscible alcohols is highly desirable to achieve phase separation. After a single wash step, the majority of 3-HP may be recovered from the solid cake. The amount of 3-HP recovered may be at least 50%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, or at least 95% of the total 3-HP in the solids cake. There may be some residual salts remaining in the 3-HP stream so obtained. The amount of non-3-HP salts remaining in the 3-HP wash stream may be at least 0.5%, at least 1.0%, at least 1.5%, at least 2.0%, at least 2.5%, at least 3.0%, at least 3.5%, at least 4.0%, at least 4.5%, at least 5.0%, at least 6.0%, at least 7.0%, at least 8.0%, at least 9.0%, at least 10.0%, at least 11.0%, at least 12.0%, at least 13.0%, at least 14.0%, at least 15.0%, at least 18.0%, at least 20.0%, at least 25.0%, or at least 30.0% of the total stream mass. The wash stream may be used in a subsequent primary extraction to reduce solvent usage. The washed salts may be relatively pure and may be a valuable nitrogen source for agriculture upon drying.

The purified 3-HP extract resulting after acidification and separation of the solid cake may be extracted into an organic solvent such as an alcohol. Other solvents may be employed when potential ester formation is not desired. The following paragraphs focus on use of an alcohol solvent. An alcohol may be selected from C₁-C₁₂ aliphatic alcohols, for example but not limited to, methanol, ethanol, 1-propanol, 1-butanol, isopropyl alcohol, isobutyl alcohol, t-butanol, 1-octanol, 1-hexanol, and their isomers. A selected alcohol may be an alcohol which is miscible or immiscible with water. This is in contrast to traditional biphasic extraction techniques in which water-immiscible alcohols is desirable. The amount of alcohols used should be enough to achieve desirable extraction efficiency. On the other hand, to minimize cost and environmental impact, it is desirable to use as a small amount as possible. Optimization may be required to achieve a right balance. In some embodiments, about 5 mol equivalents of an alcohol solvent based on the amount of 3-HP may be used. In some further embodiments, less than 2 mol equivalents of an alcohol based on the amount of 3-HP may be used. In a particular embodiment, about 1 mol equivalent of an alcohol based on the amount of 3-HP may be used. The amount of alcohol solvent used may also be calculated based on the volume of purified 3-HP. In some embodiments, a 3× volume of alcohol may be used. The extraction may be carried out in single stage or it may be carried out in multiple stages to improve extraction efficiency. The number of stages could depend on the type of extractor being used. For example, a Karr column or a Scheibel column may be used for extraction. In some embodiments, a single stage extraction may be used. In some other embodiments, two separate stages may be used. In yet some other embodiments, three separate stages may be used. The extraction efficiency may depend on the nature and/or the amount of alcohol solvent used and/or the number of stages used for the extraction. The efficiency may be at least 50%, at least 60%, at least 70%, at least 75%, at least 80%, at least 85%, at least 90%, at least 91%, at least 92%, at least 93%, at least 94%, at least 95%, at least 96%, at least 97%, or at least 98%. The efficiency may be optimized and improved using a multi-stage counter current extractor or other staged type separation systems. The heavy phase, which results from the removal of 3-HP and other organic acids, is a combination of sugars and salts. It is insoluble in alcohols and may contain some 3-HP. The amount of 3-HP in the heavy phase may be about 0.5%, about 1%, about 2%, about 3%, about 4%, about 5%, about 6%, about 7%, about 8%, about 9%, or about 10%. Upon drying, this side stream may be high in carbon and may be valuable for recycle or as a sugar source.

The 3-HP extract may contain between 200-300 g/L 3-HP along with other organic acids. This range may change based on many factors including final titer in fermentation. Because water solubility in an alcohol or other organic solvent is dependent on the carbon chain length various degrees of water and associated acid may result with the choice of alcohol or other organic solvent. 3-HP extract at this point may be purified. 3-HP may be purified by distillation. 3-HP may be purified by back-extracting into water. Additionally, 3-HP may be precipitated as a salt by adjusting pH upwards to the salt form of the acid. Crystals can be obtained by the following technique/s: concentration by solvent evaporation, cooling, or the addition of a forcing solvent. The 3-HP extract may be transferred to an esterification reactor in step 106 and carry out an esterification reaction in step 107 without further purification to produce 3-HP ester. Esterification is a reaction in organic chemistry in which, typically, a carboxylic acid is reacted with an alcohol to give an ester. The reaction is usually accelerated by heating and/or catalysis. Commonly employed catalysts include acids, Lewis Acids or dehydrating reagents. The acid may be organic and inorganic acid. The Lewis acid is a substance which can employ a lone pair of electrons from another molecule in completing the stable group of one of its own atoms. The dehydrating agent includes any reagent which can facilitate a dehydration reaction, for example but not limited to, molecular sieves of varying grade. The conversion of 3-HP to its ester may be at least 90%, at least 91%, at least 92%, at least 93%, at least 94%, at least 95%, at least 96%, at least 97%, at least 98%, or at least 99%. For example, when n-butanol is used as the extraction solvent and sulfuric acid is used as the acid to adjust PH, enough acid may be present in the alcohol extract such that no extra acid is required for the esterification reaction. Additional acid may be required to drive the esterification upon the use of longer chain alcohols under other conditions. The amount could be in a range of 1 mol % to 99 mol % based on the amount of 3-HP in the extract. In the case n-butanol is used as the solvent and sulfuric acid is used to adjust pH of the concentrated fermentation extract, the alcohol extract is refluxed at the boiling point of the alcohol (118° C. in this case with butanol) in the presence of a water trap. In some cases, a phase separation trap may be used to remove the water prior to the distillate returning to the reaction vessel. In some other cases, molecular sieves may be used alone or in combination with an acid to remove the water and drive the esterification reaction to completion. The process may be optimized using about an equal molar ratio of alcohol to 3-HP which eliminates the need for solvent recycle because the extraction solvent is used up as a reactant. The increased volatility of the ester can then be used to distill the ester away from other contaminants, thus yielding high purity 3-HP ester. In some embodiments, the purity of 3-HP ester is higher than 90%. In a further embodiment, the purity of 3-HP ester is higher than 95%.

3-HP ester is a highly valuable intermediate for producing other products. For example, without being limiting, dehydration of 3-HP ester would lead to acrylic ester, which is an important class of monomer for producing polyacrylic polymers and co-polymers. Further, they can be processed to 3-HP amide using ammonia gas or ammonium ions in a trans-amidation type reaction. Dehydration of 3-HP amide could give acrylamide (step 108), which is used in producing polyacrylamide. Polyacrylamide has found broad applications in water purification, oil-drilling, gel electrophoresis, papermaking, ore processing, and the manufacture of permanent press fabrics.

As used in this specification and the appended claims, the singular forms “a,” “an,” and “the” include plural referents unless the context clearly indicates otherwise. Thus, for example, reference to “microorganism” includes a single microorganism as well as a plurality of microorganisms; and the like.

Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications, and other publications that are herein incorporated by reference, the definition set forth in this specification prevails over the definition that is incorporated herein by reference.

Certain particular embodiments of the present invention will be described in more detail, including reference to the accompanying figure(s) and table(s). The figure(s) is/are understood to provide representative illustration of the invention and are not limiting in their content or scale. It will be understood by one of ordinary skill in the art that the scope of the invention extends beyond the specific embodiments depicted. This invention also incorporates routine experimentation and optimization of the methods, apparatus, and systems described herein.

As used herein the term “about” refers to ±10% and includes ±1% and ±0.1%. The term “comprising” (and related terms such as “comprise” or “comprises” or “having” or “including”) is not intended to exclude that other certain embodiments, for example an embodiment of any composition of matter, composition, method, or process, or the like, described herein, may “consist of” or “consist essentially of” the described features.

### Fermentation Broth

The term “fermentation broth” generally refers to a mixture derived from a microbial fermentation procedure. The fermentation broth may be a mixture obtained from a microbial fermentation procedure without any purification or separation. Alternatively, the fermentation broth may be a mixture obtained from a microbial fermentation procedure after purification or separation. The fermentation broth may contain whole cells or may be substantially whole-cell free. Without being limiting, methods of purification or separation a fermentation broth prior to removing water include filtration, precipitation and centrifuge. Additionally, the fermentation broth may be treated to release 3-HP from cells. The treatment may be a lysing step.

The fermentation broth contains 3-HP. A variety of microbial systems for producing 3-HP have been described in the art, for example, US application 2011/0125118, US application 2008/0199926, and U.S. Pat. No. 6,852,517, which are herein incorporated by reference for their teaching of 3-HP production pathways and methods of microbial 3-HP production. It is understood that these references and the following discussion provide exemplary examples to which the present invention can be applied. They are meant to be illustrative. As one of ordinary skill in the art can understand, the present invention can be applied to a variety of microbial systems which produce 3-HP and related compounds.

The microbial systems may comprise a carbon source, one or more microorganism, and suitable media and culture conditions. The fermentation may be carried out in a bio-production reactor. After fermenting for a certain period of time, the crude cell broth obtained may be further processed to yield high purity 3-HP or downstream products.

The carbon source may be suitable for the intended metabolic pathway. Suitable carbon source may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrates may also be one-carbon substrates such as carbon dioxide, carbon monoxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity.

The microorganism may have one or more natural, introduced, or enhanced 3-HP bio-production pathways. The microorganism may comprise an endogenous 3-HP production pathway. The endogenous 3-HP production pathway may be enhanced to increase 3-HP production. On the other hand, the microorganism may not comprise an endogenous 3-HP production pathway. In this case, the pathway can be introduced through, for example, genetic engineering. A microorganism may be selected from bacteria, cyanobacteria, filamentous fungi, and yeasts. Since 3-HP produced during fermentation may be toxic to the microorganism used in the process, the microorganism may further comprise tolerance aspects. Such microorganisms may include, but are not limited to, any gram negative organisms, more particularly a member of the family Enterobacteriaceae, such as E. coli, or Oligotropha carboxidovorans, or Pseudomononas sp.; any gram positive microorganism, for example Bacillus subtilis, Lactobaccilus sp. or Lactococcus sp.; a yeast, for example Saccharomyces cerevisiae, Pichia pastoris or Pichia stipitis; and other groups or microbial species. More particularly, suitable microbial hosts for the bio-production of 3-HP generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Hosts that may be particularly of interest include: Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

There may be a variety of pathways and/or mechanisms to increase 3-HP production, for example, reducing the activity of fatty acid synthase and/or enhancing the activity of malonyl-CoA reductase. The modulation of the pathways can be achieved in a variety of means described in the art, such as Application number PCT/US2010/050436, published Mar. 31, 2011 as WO/2011/038364; Application number PCT/US/057690, published May 26, 2011 as WO/2011/063363, and Application number PCT/US2011/022790 published Aug. 4, 2011 as WO/2011/094457. Also incorporated by reference for the teachings of particular enzymes and metabolic pathways are U.S. Pat. No. 7,943,362 and US Patent Application No. US2011/0183391 A1, incorporated by reference for such teachings. For example, genetic engineering may be used. In addition, one or more additives may be added to the cell culture to modulate fatty acid synthase or malonyl-CoA reductase to increase the production of 3-HP.

In addition to an appropriate carbon source, such as selected from one of the herein-disclosed types, bio-production media must contain suitable minerals, salts, cofactors, buffers and other components, known to those skilled in the art, suitable for the growth of the cultures and promotion of the enzymatic pathway necessary for 3-HP production, or other products.

Typically cells are grown at a temperature in the range of about 25° C. to about 40° C. in an appropriate medium comprising water, as well as up to 70° C. for thermophilic microorganisms. Suitable growth media in the present invention are common commercially prepared media such as Luria Bertani (LB) broth, M9 minimal media, Sabouraud Dextrose (SD) broth, Yeast medium (YM) broth, yeast synthetic minimal media (Ymin), and minimal media as described herein, such as M9 minimal media. Other defined or synthetic growth media may also be used, and the appropriate medium for growth of the particular microorganism will be known by one skilled in the art of microbiology or bio-production science. In various embodiments a minimal media may be developed and used that does not comprise, or that has a low level of addition of various components, for example less than 10, 5, 2 or 1 g/L of a complex nitrogen source including but not limited to yeast extract, peptone, tryptone, soy flour, corn steep liquor, or casein. These minimal medias may also have limited supplementation of vitamin mixtures including biotin, vitamin B12 derivatives of vitamin B12, thiamin, pantothenate and other vitamins. Minimal medias may also have limited simple inorganic nutrient sources containing less than 28, 17, or 2.5 mM phosphate, less than 25 or 4 mM sulfate, and less than 130 or 50 mM total nitrogen.

Bio-production media must contain suitable carbon substrates for the intended metabolic pathways. As described hereinbefore, suitable carbon substrates may include carbon monoxide, carbon dioxide, various monomeric and oligomeric sugars, amines, and amino acids.

Suitable pH ranges for the bio-production are between pH 3.0 to pH 10.0, where pH 6.0 to pH 8.0 is a typical pH range for the initial condition. However, the actual culture conditions for a particular embodiment are not meant to be limited by these pH ranges.

Bio-productions may be performed under aerobic, microaerobic, or anaerobic conditions, with or without agitation and with or without external heating or cooling.

The amount of 3-HP or other product(s) produced in a bio-production media generally can be determined using a number of methods known in the art, for example, high performance liquid chromatography (HPLC), gas chromatography (GC), or GC/Mass Spectroscopy (MS).

Any of the microorganisms as described and/or referred to herein may be introduced into an industrial bio-production system where the microorganisms convert a carbon source into 3-HP in a commercially viable operation. The bio-production system includes the introduction of such a microorganism into a bioreactor vessel, with a carbon source substrate and bio-production media suitable for growing the microorganism, and maintaining the bio-production system within a suitable temperature range (and dissolved oxygen concentration range if the reaction is aerobic or microaerobic) for a suitable time to obtain a desired conversion of a portion of the substrate molecules to 3-HP. The fermentation process may be monitored by measuring the concentration of 3-HP in crude fermentation broth. Industrial bio-production systems and their operation are well-known to those skilled in the arts of chemical engineering and bioprocess engineering.

Bio-productions may be performed under aerobic, microaerobic, or anaerobic conditions, with or without agitation. The operation of cultures and populations of microorganisms to achieve aerobic, microaerobic and anaerobic conditions are known in the art, and dissolved oxygen levels of a liquid culture comprising a nutrient media and such microorganism populations may be monitored to maintain or confirm a desired aerobic, microaerobic or anaerobic condition.

### Separation Techniques

A variety of separation techniques may be applied for the purification of 3-HP from crude cell broth including, but are not limited to, centrifugation, evaporation, boiling, distillation, filtration, extraction, washing, crystallization, and precipitation. The techniques cited herein are meant to be illustrative and are well known in the art. Each technique may be used alone or in any combination or may be used once or multiple times. Instruments or apparatus for carrying out the purification techniques mentioned herein are well known in the art and may be commercially available in different shapes and sizes. Without being limiting, some aspects of selected techniques are described herein.

Centrifugation involves the use of a centrifugal force for the sedimentation of mixtures, typically with a centrifuge. When there are multiple components in the mixture, controlling the rate and length of the centrifugation may lead to successive separation of different components. The rate of centrifugation may be at least 250 G, at least 500 G, at least 1,000 G, at least 1,500 G, at least 2,000 G, at least 2,500 G, at least 3,000 G, at least 3, at least 500 G, at least 4,000 G, at least 4,500 G, at least 5,000 G, at least 5,500 G, at least 6,000 G, at least 6,500 G, at least 7,000 G, at least 8,000 G, at least 9,000 G, at least 10,000 G, at least 12,000 G, at least 14,000 G, at least 16,000 G, at least 20,000 G, at least 30,000 G, at least 40,000 G, at least 50,000 G, or at least 100,000 G. The length of centrifugation may be at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 12 minutes, at least 14 minutes, at least 15 minutes, at least 18 minutes, at least 20 minutes, at least 25 minutes, at least 30 minutes, at least 35 minutes, at least 40 minutes, at least 60 minutes, at least 80 minutes, at least 100 minutes, at least 200 minutes, at least 300 minutes, at least 400 minutes, at least 500 minutes, at least 600 minutes, at least 800 minutes, at least 1,000 minutes, at least 2,000 minutes, at least 3,000 minutes, at least 4,000 minutes, 5,000 minutes, at least 6,000 minutes, or at least 7,000 minutes. Centrifuges are commercially available and well known in the art.

Evaporation is the process of vaporizing a liquid, typically from the surface. Evaporation may be accelerated in a variety of ways to improve the rate of evaporation and/or energy efficiency including, but are not limited to, reducing pressure surrounding the liquid and/or heating the liquid. The pressure and/or the temperature chosen for the evaporation may depend on a variety of factors, such as the scale, the solvent, and the equipment for the purification. An ambient atmospheric pressure may be used. A pressure lower than an ambient atmospheric pressure may be used. The pressure may be about 0.1 mbar, about 0.5 mbar, about 1 mbar, about 2 mbar, about 3 mbar, about 4 mbar, about 5 mbar, about 6 mbar, about 7 mbar, about 8 mbar, about 9 mbar, about 10 mbar, about 12 mbar, about 15 mbar, about 18 mbar, about 20 mbar, about 25 mbar, about 30 mbar, about 35 mbar, about 40 mbar, about 45 mbar, about 50 mbar, about 55 mbar, about 60 mbar, about 65 mbar, about 70 mbar, about 75 mbar, about 80 mbar, about 90 mbar, about 100 mbar, about 120 mbar, about 150 mbar, about 200 mbar, or 300 mbar. The pressure may be in a range of 0.1-300 mbar. The temperature may be about 25° C., about 28° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., or about 150° C. The temperature may be in a range of 25-180° C. Mechanical recompression techniques may also be used for the evaporation. A mechanical recompression evaporator functions by compressing a vapor to a relatively high pressure so it can be condensed in an evaporator heat exchanger. The compression can be achieved with a positive-displacement, centrifugal, or axial flow compressor. Furthermore, the mechanical recompression may comprise single-effect recompression, multiple-effect recompression, single-stage recompression, multiple-stage recompression, and any combination thereof.

Distillation is the process of heating a liquid until it boils, and then condensing and collecting the hot vapor. Similar to evaporation, distillation can be carried out under reduced pressure and/or heating. Reducing the pressure around the liquid may reduce the boiling point of the liquid, thus facilitating distillation. The pressure may be an ambient atmospheric pressure or lower. The pressure may be about 0.1 mbar, about 0.5 mbar, about 1 mbar, about 2 mbar, about 3 mbar, about 4 mbar, about 5 mbar, about 6 mbar, about 7 mbar, about 8 mbar, about 9 mbar, about 10 mbar, about 12 mbar, about 15 mbar, about 18 mbar, about 20 mbar, about 25 mbar, about 30 mbar, about 35 mbar, about 40 mbar, about 45 mbar, about 50 mbar, about 55 mbar, about 60 mbar, about 65 mbar, about 70 mbar, about 75 mbar, about 80 mbar, about 90 mbar, about 100 mbar, about 120 mbar, about 150 mbar, about 200 mbar, or 300 mbar. The pressure may be in a range of 0.1-1,000 mbar. The temperature may be about 25° C., about 28° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., or about 150° C. The temperature may be in a range of 25-180° C.

Filtration is a technique used to separate solids from liquids by passing the mixture through a media through which mainly the liquids may pass. Filtration can be aided with or without additional solvent. After filtration, the solids may still contain some residual liquids. In some embodiments, the solids may contain by volume a ratio of about 10%, about 11%, about 12%, about 15%, about 20%, about 25%, about 30%, about 35%, about 40%, about 50%, about 60%, of the total volume of the liquids. The solids may be washed with a solvent to reduce the amount of residual liquids in the solids. After a single wash, the amount of residual liquids may be reduced by at least 20%, at least 30%, at least 40%, at least 50%, at least 60%, at least 70%, at least 80%, or at least 90%. In the present invention, after the wash, the combined recovered 3-HP may be at least 70%, at least 80%, or at least 90% of the total amount.

### Downstream Chemical and Consumer Products

Various embodiments described herein are related to the purification of 3-hydroxypropionic acid (3-HP) from a fermentation broth. This organic acid, 3-HP, may be converted to various other products having industrial uses including, but are not limited to, acrylamide, acrylic acid, esters of acrylic acid, 1,3-propanediol, and other chemicals, collectively referred to as “downstream chemical products.” In some instances the conversion is associated with the separation and/or purification steps. These downstream chemical products are useful for producing a variety of consumer products which will be described in detail herein. The methods of the present invention include steps to produce downstream products of 3-HP.

As a C3 building block, 3-HP offers much potential in a variety of chemical conversions to commercially important intermediates, industrial end products, and consumer products. For example, 3-HP may be converted to acrylic acid, acrylates (e.g., acrylic acid salts and esters), 1,3-propanediol, malonic acid, ethyl-3-hydroxypropionate, ethyl ethoxy propionate, propiolactone, acrylamide, or acrylonitrile.

Additionally, 3-HP may be oligomerized or polymerized to form poly(3-hydroxypropionate) homopolymers, or co-polymerized with one or more other monomers to form various co-polymers. Because 3-HP has only a single stereoisomer, polymerization of 3-HP is not complicated by the stereo-specificity of monomers during chain growth. This is in contrast to (S)-2-Hydroxypropanoic acid (also known as lactic acid), which has two (D, L) stereoisomers that must be considered during its polymerizations.

As will be further described, 3-HP can be converted into derivatives starting (i) substantially as the protonated form of 3-hydroxypropionic acid; (ii) substantially as the deprotonated form, 3-hydroxypropionate; or (iii) as mixtures of the protonated and deprotonated forms. Generally, the fraction of 3-HP present as the acid versus the salt will depend on the pH, the presence of other ionic species in solution, temperature (which changes the equilibrium constant relating the acid and salt forms), and to some extent pressure. Many chemical conversions may be carried out from either of the 3-HP forms, and overall process economics will typically dictate the form of 3-HP for downstream conversion.

Acrylic acid obtained from 3-HP made by the present invention may be further converted to various polymers. For example, the free-radical polymerization of acrylic acid takes place by polymerization methods known to the skilled worker and can be carried out either in an emulsion or suspension in aqueous solution or another solvent. Initiators, such as but not limited to organic peroxides, often are added to aid in the polymerization. Among the classes of organic peroxides that may be used as initiators are diacyls, peroxydicarbonates, monoperoxycarbonates, peroxyketals, peroxyesters, dialkyls, and hydroperoxides. Another class of initiators is azo initiators, which may be used for acrylate polymerization as well as co-polymerization with other monomers. U.S. Pat. Nos. 5,470,928; 5,510,307; 6,709,919; and 7,678,869 teach various approaches to polymerization using a number of initiators, including organic peroxides, azo compounds, and other chemical types, and are incorporated by reference for such teachings as applicable to the polymers described herein.

Accordingly, it is further possible for co-monomers, such as crosslinkers, to be present during the polymerization. The free-radical polymerization of the acrylic acid obtained from dehydration of 3-HP, as produced herein, in at least partly neutralized form and in the presence of crosslinkers is practiced in certain embodiments. This polymerization may result in hydrogels which can then be comminuted, ground and, where appropriate, surface-modified, by known techniques.

An important commercial use of polyacrylic acid is for superabsorbent polymers. This specification hereby incorporates by reference Modern Superabsorbent Polymer Technology, Buchholz and Graham (Editors), Wiley-VCH, 1997, in its entirety for its teachings regarding superabsorbent polymers components, manufacture, properties and uses. Superabsorbent polymers are primarily used as absorbents for water and aqueous solutions for diapers, adult incontinence products, feminine hygiene products, and similar consumer products. In such consumer products, superabsorbent materials can replace traditional absorbent materials such as cloth, cotton, paper wadding, and cellulose fiber. Superabsorbent polymers absorb, and retain under a slight mechanical pressure, up to 25 times or more their weight in liquid. The swollen gel holds the liquid in a solid, rubbery state and prevents the liquid from leaking. Superabsorbent polymer particles can be surface-modified to produce a shell structure with the shell being more highly crosslinked. This technique improves the balance of absorption, absorption under load, and resistance to gel-blocking. It is recognized that superabsorbent polymers have uses in fields other than consumer products, including agriculture, horticulture, and medicine.

Superabsorbent polymers are prepared from acrylic acid (such as acrylic acid derived from 3-HP provided herein) and a crosslinker, by solution or suspension polymerization. Exemplary methods include U.S. Pat. Nos. 5,145,906; 5,350,799; 5,342,899; 4,857,610; 4,985,518; 4,708, 997; 5,180,798; 4,666,983; 4,734,478; and 5,331,059, each incorporated by reference for their teachings relating to superabsorbent polymers.

Among consumer products, a diaper, a feminine hygiene product, and an adult incontinence product are made with superabsorbent polymer that itself is made substantially from acrylic acid converted from 3-HP made in accordance with the present invention.

Diapers and other personal hygiene products may be produced that incorporate superabsorbent polymer made from acrylic acid made from 3-HP which is bio-produced by the teachings of the present application. The following provides general guidance for making a diaper that incorporates such superabsorbent polymer. The superabsorbent polymer first is prepared into an absorbent pad that may be vacuum formed, and in which other materials, such as a fibrous material (e.g., wood pulp) are added. The absorbent pad then is assembled with sheet(s) of fabric, generally a nonwoven fabric (e.g., made from one or more of nylon, polyester, polyethylene, and polypropylene plastics) to form diapers.

More particularly, in one non-limiting process, above a conveyer belt multiple pressurized nozzles spray superabsorbent polymer particles (such as about 400 micron size or larger), fibrous material, and/or a combination of these onto the conveyer belt at designated spaces/intervals. The conveyor belt is perforated and under vacuum from below, so that the sprayed on materials are pulled toward the belt surface to form a flat pad. In various embodiments, fibrous material is applied first on the belt, followed by a mixture of fibrous material and the superabsorbent polymer particles, followed by fibrous material, so that the superabsorbent polymer is concentrated in the middle of the pad. A leveling roller may be used toward the end of the belt path to yield pads of uniform thickness. Each pad thereafter may be further processed, such as to cut it to a proper shape for the diaper, or the pad may be in the form of a long roll sufficient for multiple diapers. Thereafter, the pad is sandwiched between a top sheet and a bottom sheet of fabric (one generally being liquid pervious, the other liquid impervious), such as on a conveyor belt, and these are attached together such as by gluing, heating or ultrasonic welding, and cut into diaper-sized units (if not previously so cut). Additional features may be provided, such as elastic components, strips of tape, etc., for fit and ease of wearing by a person.

The ratio of the fibrous material to polymer particles is known to effect performance characteristics. In some cases, this ratio is between 75:25 and 90:10 (see U.S. Pat. No. 4,685,915, incorporated by reference for its teachings of diaper manufacture). Other disposable absorbent articles may be constructed in a similar fashion, such as for adult incontinence, feminine hygiene (sanitary napkins), tampons, etc. (see, for example, U.S. Pat. Nos. 5,009,653, 5,558,656, and 5,827,255 incorporated by reference for their teachings of sanitary napkin manufacture).

Low molecular-weight polyacrylic acid has uses for water treatment, flocculants, and thickeners for various applications including cosmetics and pharmaceutical preparations. For these applications, the polymer may be uncrosslinked or lightly crosslinked, depending on the specific application. The molecular weights are typically from about 200 to about 1,000,000 g/mol. Preparation of these low molecular-weight polyacrylic acid polymers is described in U.S. Pat. Nos. 3,904,685; 4,301,266; 2,798,053; and 5,093,472, each of which is incorporated by reference for its teachings relating to methods to produce these polymers.

Acrylic acid may be co-polymerized with one or more other monomers selected from acrylamide, 2-acrylamido-2-methylpropanesulfonic acid, N,N-dimethylacrylamide, N-isopropylacrylamide, methacrylic acid, and methacrylamide, to name a few. The relative reactivities of the monomers affect the microstructure and thus the physical properties of the polymer. Co-monomers may be derived from 3-HP, or otherwise provided, to produce co-polymers. Ulmann's Encyclopedia of Industrial Chemistry, Polyacrylamides and Poly(Acrylic Acids), WileyVCH Verlag GmbH, Wienham (2005), is incorporated by reference herein for its teachings of polymer and co-polymer processing.

Acrylic acid can in principle be copolymerized with almost any free-radically polymerizable monomers including styrene, butadiene, acrylonitrile, acrylic esters, maleic acid, maleic anhydride, vinyl chloride, acrylamide, itaconic acid, and so on. End-use applications typically dictate the co-polymer composition, which influences properties. Acrylic acid also may have a number of optional substitutions on it, and after such substitutions be used as a monomer for polymerization, or co-polymerization reactions. As a general rule, acrylic acid (or one of its co-polymerization monomers) may be substituted by any substituent that does not interfere with the polymerization process, such as alkyl, alkoxy, aryl, heteroaryl, benzyl, vinyl, allyl, hydroxy, epoxy, amide, ethers, esters, ketones, maleimides, succinimides, sulfoxides, glycidyl and silyl (see U.S. Pat. No. 7,678,869, incorporated by reference above, for further discussion). The following paragraphs provide a few non-limiting examples of copolymerization applications.

Paints that comprise polymers and copolymers of acrylic acid and its esters are in wide use as industrial and consumer products. Aspects of the technology for making such paints can be found in U.S. Pat. Nos. 3,687,885 and 3,891,591, incorporated by reference for its teachings of such paint manufacture. Generally, acrylic acid and its esters may form homopolymers or copolymers among themselves or with other monomers, such as amides, methacrylates, acrylonitrile, vinyl, styrene and butadiene. A desired mixture of homopolymers and/or copolymers, referred to in the paint industry as ‘vehicle’ (or ‘binder’) are added to an aqueous solution and agitated sufficiently to form an aqueous dispersion that includes sub-micrometer sized polymer particles. The paint cures by coalescence of these ‘vehicle’ particles as the water and any other solvent evaporate. Other additives to the aqueous dispersion may include pigment, filler (e.g., calcium carbonate, aluminum silicate), solvent (e.g., acetone, benzol, alcohols, etc., although these are not found in certain no VOC paints), thickener, and additional additives depending on the conditions, applications, intended surfaces, etc. In many paints, the weight percent of the vehicle portion may range from about nine to about 26 percent, but for other paints the weight percent may vary beyond this range.

Acrylic-based polymers are used for many coatings in addition to paints. For example, for paper coating latexes, acrylic acid is used from 0.1-5.0%, along with styrene and butadiene, to enhance binding to the paper and modify rheology, freeze-thaw stability and shear stability. In this context, U.S. Pat. Nos. 3,875,101 and 3,872,037 are incorporated by reference for their teachings regarding such latexes. Acrylate-based polymers also are used in many inks, particularly UV curable printing inks. For water treatment, acrylamide and/or hydroxy ethyl acrylate are commonly co-polymerized with acrylic acid to produce low molecular-weight linear polymers. In this context, U.S. Pat. Nos. 4,431,547 and 4,029,577 are incorporated by reference for their teachings of such polymers. Co-polymers of acrylic acid with maleic acid or itaconic acid are also produced for water-treatment applications, as described in U.S. Pat. No. 5,135,677, incorporated by reference for that teaching. Sodium acrylate (the sodium salt of glacial acrylic acid) can be co-polymerized with acrylamide (which may be derived from acrylic acid via amidation chemistry) to make an anionic co-polymer that is used as a flocculant in water treatment.

For thickening agents, a variety of co-monomers can be used, such as described in U.S. Pat. Nos. 4,268,641 and 3,915,921, incorporated by reference for description of these co-monomers. U.S. Pat. No. 5,135,677 describes a number of co-monomers that can be used with acrylic acid to produce water-soluble polymers, and is incorporated by reference for such description.

Also as noted, some conversions to downstream products may be made enzymatically. For example, 3-HP may be converted to 3-HP-CoA, which then may be converted into polymerized 3-HP with an enzyme having polyhydroxyacid synthase activity (EC 2.3.1.-). Also, 1,3-propanediol can be made using polypeptides having oxidoreductase activity or reductase activity (e.g., enzymes in the EC 1.1.1.-class of enzymes). Alternatively, when creating 1,3-propanediol from 3-HP, a combination of (1) a polypeptide having aldehyde dehydrogenase activity (e.g., an enzyme from the 1.1.1.34 class) and (2) a polypeptide having alcohol dehydrogenase activity (e.g., an enzyme from the 1.1.1.32 class) can be used. Polypeptides having lipase activity may be used to form esters. Enzymatic reactions such as these may be conducted in vitro, such as using cell-free extracts, or in vivo.

Thus, various embodiments of the present invention, such as methods of making a chemical, include conversion steps to any such noted downstream products of microbially produced 3-HP, including but not limited to those chemicals described herein and in the incorporated references (the latter for jurisdictions allowing this). For example, one embodiment is making 3-HP molecules by the teachings herein and further converting the 3-HP molecules to polymerized-3-HP (poly-3-HP) or acrylic acid, and such as from acrylic acid then producing from the 3-HP molecules any one of polyacrylic acid (polymerized acrylic acid, in various forms), methyl acrylate, acrylamide, acrylonitrile, propiolactone, ethyl 3-HP, malonic acid, 1,3-propanediol, ethyl acrylate, n-butyl acrylate, hydroxypropyl acrylate, hydroxyethyl acrylate, isobutyl acrylate, 2-ethylhexyl acrylate, and acrylic acid or an acrylic acid ester to which an alkyl or aryl addition is made, and/or to which halogens, aromatic amines or amides, and aromatic hydrocarbons are added.

Reactions that form downstream compounds such as acrylates or acrylamides can be conducted in conjunction with use of suitable stabilizing agents or inhibiting agents reducing likelihood of polymer formation. See, for example, U.S. Patent Publication No. 2007/0219390 A1. Stabilizing agents and/or inhibiting agents include, but are not limited to, e.g., phenolic compounds (e.g., dimethoxyphenol (DMP) or alkylated phenolic compounds such as di-tert-butyl phenol), quinones (e.g., t-butyl hydroquinone or the monomethyl ether of hydroquinone (MEHQ)), and/or metallic copper or copper salts (e.g., copper sulfate, copper chloride, or copper acetate). Inhibitors and/or stabilizers can be used individually or in combinations as will be known by those of skill in the art. Also, in various embodiments, the one or more downstream compounds is/are recovered at a molar yield of up to about 100 percent, or a molar yield in the range from about 70 percent to about 90 percent, or a molar yield in the range from about 80 percent to about 100 percent, or a molar yield in the range from about 90 percent to about 100 percent. Such yields may be the result of single-pass (batch or continuous) or iterative separation and purification steps in a particular process.

The methods of the present invention can also be used to produce downstream compounds derived from 3-HP, such as but not limited to, polymerized-3-HP (poly-3-HP), acrylic acid, polyacrylic acid (polymerized acrylic acid, in various forms), copolymers of acrylic acid and acrylic esters, acrylamide, acrylonitrile, propiolactone, ethyl 3-HP, malonic acid, and 1,3-propanediol. Also, among esters that are formed are methyl acrylate, ethyl acrylate, n-butyl acrylate, hydroxypropyl acrylate, hydroxyethyl acrylate, isobutyl acrylate, and 2-ethylhexyl acrylate. These and/or other acrylic acid and/or other acrylate esters may be combined, including with other compounds, to form various known acrylic acid-based polymers. Numerous approaches may be employed for such downstream conversions, generally falling into enzymatic, catalytic (chemical conversion process using a catalyst), thermal, and combinations thereof (including some wherein a desired pressure is applied to accelerate a reaction). For example, without being limiting, acrylic acid may be made from 3-HP via a dehydration reaction, methyl acrylate may be made from 3-HP via dehydration and esterification, the latter to add a methyl group (such as using methanol), acrylamide may be made from 3-HP via dehydration and amidation reactions, acrylonitrile may be made via a dehydration reaction and forming a nitrile moiety, propriolactone may be made from 3-HP via a ring-forming internal esterification reaction, ethyl-3-HP may be made from 3-HP via esterification with ethanol, malonic acid may be made from 3-HP via an oxidation reaction, and 1,3-propanediol may be made from 3-HP via a reduction reaction. Additionally, it is appreciated that various derivatives of the derivatives of 3-HP and acrylic acid may be made, such as the various known polymers of acrylic acid and its derivatives. Production of such polymers is considered within the scope of the present invention. Copolymers containing acrylic acid and/or esters have been widely used in the pharmaceutical formulation to achieve extended or sustained release of active ingredients, for example as coating material. Downstream compounds may also be converted to consumer products such as diapers, carpet, paint, and adhesives.

Another important product, acrylamide, has been used in a number of industrial applications. Acrylamide may be produced from 3-HP, for example, without being limiting, via an esterification-amidation-dehydration sequence. Refluxing an alcohol solution of 3-HP in the presence of an acid or Lewis acid catalyst described herein would lead to a 3-HP ester. Treatment of the 3-HP ester with either an ammonia gas or an ammonium ion could yield 3-HP amide. Finally, dehydration of the 3-HP amide with dehydration reagents described earlier in this application could produce acrylamide. The steps mentioned herein may be rearranged to produce the same final product acrylamide. Polymerization of acrylamide can be achieved, for example and without being limiting, by radical polymerization. Polyacrylamide polymers have been widely used as additives for treating municipal drinking water and waste water. In addition, they have found applications in gel electrophoresis, oil-drilling, papermaking, ore processing, and the manufacture of permanent press fabrics.

### Solvent

A variety of solvents may be used in the present invention as long as 3-HP is soluble in the chosen solvent. A solvent may be selected such that other impurities in a fermentation broth are less soluble in the solvent than 3-HP. In some embodiments, the solvent comprises C₁-C₁₂ alcohols. In a further embodiment, the alcohols are aliphatic alcohols. The aliphatic alcohols may be primary, secondary, or tertiary alcohols. They may be linear or branched alcohols. They may be miscible or immiscible with water. Their boiling points may be in a range from about 30° C. to about 150° C. The choice of alcohol solvent may depend on many factors, for example but not limited to, the scale of the reaction, the product desired, and the reactor used. Non-alcohol organic solvents such as ethers, ketones or aldehydes may be used as extracting solvents for 3-HP when esterification reactions are not wanted. For example if 3HP is the intended isolate. Non-limiting examples of such solvents are tetrahydrofuran, methyl ethyl ketone, methyl aldehyde, ethyl aldehyde, diethyl ether, propyl aldehyde and acetone.

### Acid

In the present disclosure, an acid may be used to adjust PH and/or a catalyst to accelerate an organic transformation, such as an esterification reaction. Suitable acid include acidic resins, acidic inorganic salts, and mineral acids. Non-limiting examples of mineral acids include sulfuric acid, hydrochloric acid, polyphosphoric acid, phosphoric acid and hydrobromic acid. Non-limiting examples of inorganic salts include copper sulfate, FeCl₃ and AlCl₃. Non-limiting examples of acidic resins include AMBERLYST® resin, NAFION™ resins and acidic DOWEX™ resins.

After the addition of an acid, the pH of the mixture may be in a range of less than 0, less than 1, less than 2, less than 3, less than 4, less than 5, less than 6, less than 7 or less than 8. Alternatively, the pH may be about 0, 1, 1.5, 2.0, 2.5, 3.0, 3.5, 4.0, 4.5, 5.0, 5.5, 6.0, 6.6 or 7.

### EXAMPLES

The examples below illustrate the invention without in any way limiting its scope.

### Example 1

### Step 101:

A neutral pH fermentation broth was centrifuged (for example at 3250 G or greater) for at least 5 minutes to remove whole cells.

### Step 102:

Water in the clarified fermentation broth was removed by evaporation. This was conducted by decreasing the pressure and/or heating the solution. A pressure of 60 mbar at 60° C. was effective. In addition, an atmospheric pressure at 100° C. was also effective. The distilled water could then be re-used in the fermentation process.

### Step 103:

After the removal of water, precipitated solids such as salts and proteins were separated either during the concentration using a scraped drum evaporator or after the concentration in a separate step using centrifugation or filtration to give a clarified concentrated fermentation broth. The solids obtained were washed with an organic solvent such as an alcohol to improve the recovery of 3-HP.

### Step 104:

The clarified concentrated fermentation broth was acidified with sulfuric acid to give an acidified concentrated fermentation broth of, for example, about pH=2. Solids precipitated were collected by filtration. The salt cake obtained was washed with an alcohol, for example, butanol. Over 90% of the 3-HP was recovered from the salt cake after a single wash step.

### Step 105:

The acidified concentrated fermentation broth was extracted into a 3× volume of the alcohol, for example, butanol in 3 separate stages to achieve greater than 95% extraction efficiency. Upon complete extraction of 3-HP into the alcohol, a separate solid or viscous phase remained. The extraction represented a purification of the 3-HP away from organic insoluble impurities.

### Step 106:

The 3-HP alcohol extract contained between 200-300 g/13-HP along with other organic acids. When butanol was used for an extraction solvent, enough acid was extracted into butanol that no extra acid was required for the esterification reaction. The butanol extract was refluxed at 118° C. in the presence of a water trap to generate 3-HP butyl ester.

### Step 107:

The 3-HP ester can be further processed to 3-HP amide using ammonia gas or an ammonium ion in an amidation type reaction. The use of an ester of 3-HP for the amidation reaction was advantageous over that of 3-HP in the acid form since the ester group is more reactive towards nitrogen-based nucleophiles than the carboxylic acid group.

### Step 108:

The conversion of 3-HP amide via dehydration to acrylamide can be conducted using heat and/or catalyst with or without an external solvent. The temperature may be in a range of 25° C. to 250° C. The catalyst may be an acid such as a Lewis acid, an inorganic acid, or an organic acid, or a base such as a hydroxide or an amine, either organic or inorganic. The amount of catalyst could be in a range of about 0.1% to about 99%. In addition, a dehydration reagent, such as molecular sieves or ortho-esters, may be added to facilitate the dehydration reaction.

### Example 2

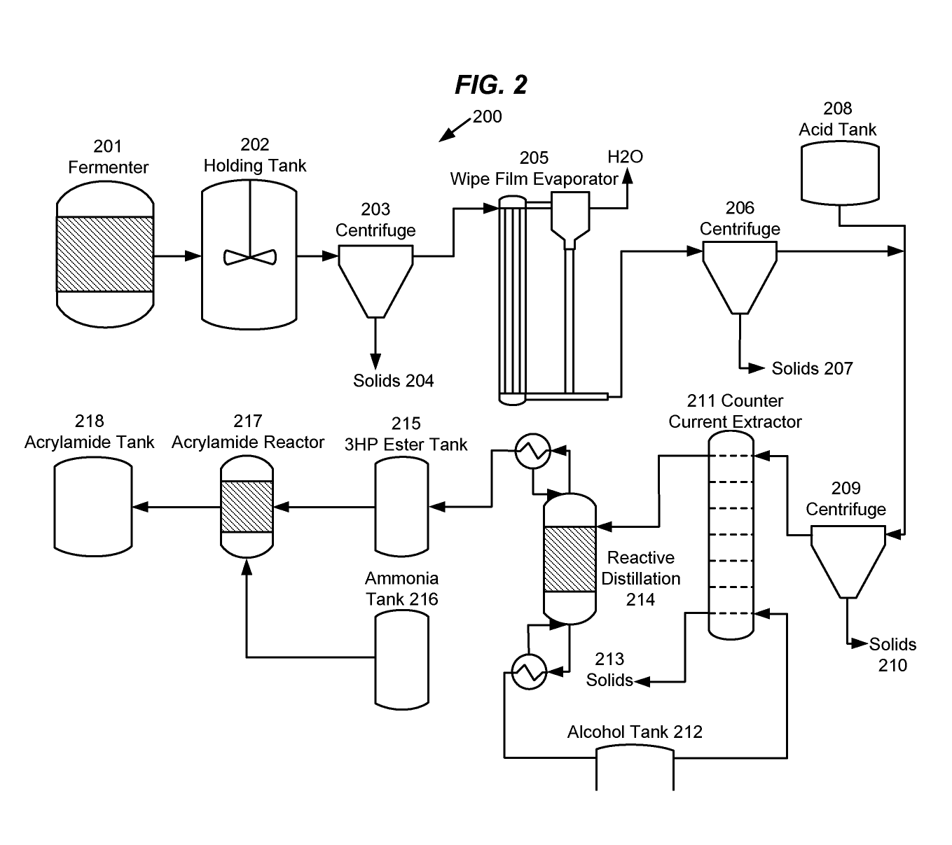

FIG. 2 outlines an example of producing 3-HP with system 200. System 200 includes a fermenter 201, a holding tank 202, three centrifuges 203, 206 and 209, a wiped film evaporator 205, an acid tank 208, a counter current extractor 211, an alcohol tank 212, a reactive distillation apparatus 214, a 3-HP ester tank 215, an ammonia tank 216, an acrylamide reactor 217 and an acrylamide tank 218. Although various components of system 200 are described below and depicted in FIG. 2, the components need not necessarily all be present, and in some cases may be present in a different order than the order shown in FIG. 2.

3-HP is produced by bacterial cells during a fermentation process in the fermenter 201. The concentration of 3-HP may be in a range of 2-200 g/L, or at least 10 g/L. The whole cell broth from the fermentation process is transferred to the holding tank 202 prior to feeding into the centrifuge 203. The broth is centrifuged at 3250 G for 5 minutes or 1 million G for less than 1 minute. After removing solids 204, the remaining liquid is transferred to a wiped film evaporator 205. Water is evaporated at skin temperature of 135° C., vacuum of 25 mmHg and RPM (revolutions per minute) of 250. The water is recycled and reused in the fermentation process. The concentrated fermentation broth contains 3-HP at a concentration of at least 50-200 g/L, such as at least 100 g/L. The concentrated broth is fed into the second centrifuge 206 to remove solids 207. Thereafter, a clarified concentrated fermentation broth is obtained. The pH of the clarified concentrated fermentation broth is adjusted with an acid from the acid tank 208. An inorganic acid, for example, sulfuric acid or phosphoric acid, may be used. Insoluble salts 210 are further removed using the third centrifuge 209. The 3-HP in the acidified clarified concentrate is extracted into an alcohol using the counter current extractor 211. Esterification is conducted in the reactive distillation apparatus 214 and the resulting 3-HP ester is distillated off and collected in 3-HP ester tank 215. The 3-HP ester undergoes an amidation and dehydration reaction in the amidation reactor 217 to produce acrylamide. The amidation reaction is carried out with ammonia gas from the ammonia tank 216. Thereafter, the resulting acrylamide is collected and/or stored in acrylamide tank 218. The production output of acrylamide may be at least 1, 2, 3, 4, 5, 6, 7, 8, 9, 10, 20, 30, 40, 50, 60, 70, 80, 90, 100, 120, 150, 200, 300 or 500 tons per day.

While various embodiments of the present invention have been shown and described herein, it will be obvious to those skilled in the art that such embodiments are provided by way of example only. Numerous variations, changes, and substitutions will now occur to those skilled in the art without departing from the invention. It is intended that the following claims define the scope of the invention and that methods and structures within the scope of these claims and their equivalents be covered thereby.

What is claimed is:

1. A process for producing high purity 3-hydroxypropionic acid (3-HP), comprising: (a) providing a fermentation broth comprising 3-HP, or salt thereof; (b) removing a substantial amount of water from said fermentation broth to give a concentrated fermentation broth; (c) extracting said 3-HP from said concentrated fermentation broth with an organic solvent, wherein said organic solvent has a boiling point of less than 170° C.; and (d) distilling the extract from step (c) to give said high purity 3-HP and/or its ester.

2. The process of claim 1, wherein said high purity 3-HP has purity higher than 90%.

3. The process of claim 1, wherein said high purity 3-HP has purity higher than 95%.

4. The process of claim 1, wherein a substantial amount of whole cells are removed from said fermentation broth prior to step (a).

5. The process of claim 1, wherein a substantial amount of whole cells are removed from said fermentation broth after step (a).

6. The process of claim 4 or 5, wherein said substantial amount of whole cells are removed with a centrifuge.

7. The process of claim 1, wherein said removing a substantial amount of water is performed by evaporation.

8. The process of claim 1, wherein said evaporation is carried out at a temperature range of 50-150° C.

9. The process of claim 1, wherein said evaporation is carried out at a reduced pressure of 20-1,000 mbar.

10. The process of claim 1, wherein said removing a substantial amount of water is performed with mechanical recompression methods.

11. The process of claim 1, wherein said removing a substantial amount of water is performed with a thin film evaporator.

12. The process of claim 1, wherein at least 80% of water in said fermentation broth is removed.

13. The process of claim 1, wherein at least 90% of water in said fermentation broth is removed.

14. The process of claim 1, wherein at least 95% of water in said fermentation broth is removed.

15. The process of claim 1, wherein at least 99% of water in said fermentation broth is removed.

16. The process of claim 1, further comprising separating any resulting solids during or after removing water.

17. The process of claim 1, further comprising adjusting pH of said concentrated fermentation broth with an acid after step (b).

18. The process of claim 17, wherein said acid is sulfuric acid.

19. The process of claim 17, wherein said acid is phosphoric acid.

20. The process of claim 17, further comprising separating any resulting solids during or after the addition of said acid.

21. The process of claim 1, wherein said organic solvent is an alcohol solvent.

22. The process of claim 21, wherein said alcohol solvent is selected from the group consisting of C₁-C₁₂ aliphatic alcohols.

23. The process of claim 21, wherein said alcohol solvent is methanol.

24. The process of claim 21, wherein said alcohol solvent is selected from ethanol and propanol.

25. The process of claim 21, wherein said alcohol solvent is butanol.

26. The process of claim 21, wherein said alcohol solvent is hexanol.

27. The process of claim 1, wherein said organic solvent is selected from the group consisting of aldehydes, ketones and ethers.

28. The process of claim 1, wherein about 90% of 3-HP is recovered from said concentrated fermentation broth after step (b).

29. The process of claim 21, further comprising refluxing and/or distilling the 3-HP containing extract to generate a 3-HP ester.

30. The process of claim 29, wherein refluxing comprises adding a catalytic amount of acid.

31. The process of claim 30, wherein said acid is sulfuric acid.

32. The process of claim 29, further comprising carrying out an amidation reaction to generate a 3-HP amide.

33. The process of claim 32, further comprising a dehydration reaction to generate acrylamide.

34. The process of claim 29, wherein less than two equivalents of said alcohol solvent based on the amount of 3-HP is used to produce said 3-HP ester.

35. The process of claim 34, wherein about one equivalent of said alcohol solvent based on the amount of 3-HP is used to produce said 3-HP ester.

36. A high purity 3-HP produced by the steps of: (a) providing a fermentation broth comprising 3-HP, or salt thereof; (b) removing a substantial amount of water from said fermentation broth to give a concentrated fermentation broth; (c) extracting said 3-HP from said concentrated fermentation broth with an organic solvent; and (d) distilling said 3-HP to give said high purity 3-HP.

37. A 3-HP ester produced by the steps of: (a) providing a fermentation broth comprising 3-HP, or salt thereof; (b) removing a substantial amount of water from said fermentation broth to give a concentrated fermentation broth; (c) extracting said 3-HP from said concentrated fermentation broth with an alcohol solvent; and (d) refluxing and/or distilling the 3-HP containing extract to generate said 3-HP ester.

38. 3-HP amide produced by the steps of: (a) providing a fermentation broth comprising 3-HP, or salt thereof; (b) removing a substantial amount of water from said fermentation broth to give a concentrated fermentation broth; (c) extracting said 3-HP from said concentrated fermentation broth with an organic solvent; and (d) converting said 3-HP to 3-HP amide.

39. Acrylamide produced by the steps of: (a) providing a fermentation broth comprising 3-HP, or salt thereof; (b) removing a substantial amount of water from said fermentation broth to give a concentrated fermentation broth; (c) extracting said 3-HP from said concentrated fermentation broth with an organic solvent; and (d) converting said 3-HP to acrylamide.

40. A process of producing a 3-HP-based product, comprising:
(a) producing 3-HP according to the process of claim 1; and
(b) converting said 3-HP into a 3-HP-based product.

41. The product of claim 40, wherein the product is selected from the group consisting of a diaper, a feminine hygiene product, an adult incontinence product, a paint, a coating, an ink, and a thickening agent.

42. A system, comprising: (a) a fermenter; (b) an evaporator; (c) a centrifuge; (d) an extractor; (e) a reactive distillation apparatus; (f) an esterification tank; and (g) an amidation reactor.

43. The system of claim 42, wherein said evaporator is upstream of said extractor.

44. The system of claim 42, wherein water is removed by said evaporator.

45. The system of claim 42, wherein said fermenter comprises a fermentation broth having a 3-HP concentration of at least 10 g/L.

46. The system of claim 42, wherein said centrifuge comprises a liquid phase having a 3-HP concentration of at least 10 g/L.

47. A process for purifying 3-HP from a fermentation broth, comprising: (a) removing a substantial amount of water from said fermentation broth to give a concentrated fermentation broth; and (b) extracting said 3-HP from said concentrated fermentation broth with an organic solvent.
